(19) United States
(12) Patent Application Publication
(10) Pub. No.: US 2009/0099480 A1
(43) Pub. Date: Apr. 16, 2009

(54) System and method for patient monitoring

(76) Inventors: Peter Salgo, New York, NY (US); Robert Golden, Morning View, KY (US)

Correspondence Address: GOODWIN PROCTER LLP;ATTN: PATENT ADMINISTRATOR, 620 Eighth Avenue, NEW YORK, NY, 10018, US

(21) Appl. No.: 12/154,559

(22) Filed: May 23, 2008

Related U.S. Application Data
(60) Provisional application No. 60/931,606, filed on May 24, 2007.

(51) Int. Cl. A61B 5/103

(52) U.S. Cl. 600/595

(57) ABSTRACT

The present invention relates to a patient monitoring system for automatically monitoring patient parameters over time while the patient occupies a bed. The patient monitoring system may include a sensing system positioned underneath a patient and separated from the patient by at least one layer of material, the sensing system comprising a plurality of sensor cells, the sensor cells automatically collecting sensor data related to the patient parameters. The patient monitoring system may further include an interface for receiving the collected sensor data from the sensing system and a monitoring engine receiving the collected sensor data from the interface. Calculation components may be provided for determining the patient parameters from the collected sensor data.

### CROSS REFERENCE TO RELATED APPLICATIONS

This application claims priority to U.S. Provisional Application Ser. No. 60/931,606 filed on May 24, 2007 the entirety of which is incorporated herein by reference.

### TECHNICAL FIELD

The present invention is related generally to systems and methods for patient monitoring and, more particularly, to a system and method for continuously and remotely monitoring various patient parameters.

### BACKGROUND OF THE INVENTION

Although there are a variety of known approaches to patient monitoring, these approaches have met with only limited success. In view of the information that may be gathered through continuous patient monitoring and the medical benefits that may be produced with the use of this information, it is highly desirable to provide improved techniques for monitoring and detecting various patient parameters. Knowledge of such parameters as pressure, weight, activity, and position is valuable for maintaining and improving a patient's medical condition.

Currently implemented systems often require constant staff supervision to monitor patient parameters such as pressure, weight, activity, and position. Furthermore, multiple systems are required to monitor all of these parameters and each system may be exceedingly expensive.

The pressure a patient's body is exerting on the bed, the distribution and magnitude of the pressure points, and the duration of high pressure levels in the absence of movement are the causes of one of the most severe healthcare problems. High pressures at one location for extended times are the cause of decubitus ulcers, otherwise known as bedsores. Bedsores result in astounding numbers of amputations and fatalities. Every year the problem worsens as the older segment of the population grows. Experts agree that prevention is vastly more cost-effective than treatment.

With regard to weight, knowing a patient's weight, particularly a bedridden patient's weight, is critical for determining patient treatments. For example, determining correct medication doses requires knowledge of patient weight as most medications are prescribed in milligrams per kilogram of body weight (mg/kg). Lack of knowledge regarding a patient's weight result in an incorrect dosage. Furthermore, fluctuations in patient weight are the key indicators of fluid and food intake and outtake. Imbalances in either are indications of failing health. Because many patients can't leave their beds to be weighed, clinicians often guess their patients' weight.

With regard to activity, the range of human activity in bed from comatose to quiet, motionless through restless to convulsing and finally to “missing” is important and difficult to record, quantify, and observe. Beyond simple observation, knowing and establishing alerts based upon the level of agitation or quiescence is a key element of responsible patient care. This is especially true for elderly, mentally-impaired, surgical recovery and crisis-point patients. All of these patients are at risk of becoming dangerously hyperactive or inactive if not monitored continuously. Injuries from falling are a well-known problem that has escalated with an aging population and the increasing unacceptability of patient restraints. Bed rails, restraints and other physical techniques of preventing a patient from falling have proven dangerous and are often rejected by patients.

Related to the monitoring of activity is the monitoring of physical position. Certain physical positions, particularly for surgical, orthopedic, and cognitively impaired patients, are undesirable. Positions that constrict blood flow put unacceptable amounts of strain on healing areas, or cause discomfort. As such, changes in position need to be monitored and sometimes averted.

Monitoring these factors is highly important, but obtaining a momentary reading of them can be meaningless. One of the critical tasks of patient care is the recording of the patient's condition over time. Detecting a single instance of any key parameter without knowing the history and trends associated with it can be valueless.

Currently available solutions for monitoring the above-identified parameters have proven inadequate. For example, for relieving pressure, pressure adjusting beds have been provided for adjusting pressure and preventing bedsores in the absence of adequate monitoring. These beds are an extremely expensive alternative to proper patient monitoring and, due to cost, are available to only a small percentage of the patient population at risk.

For monitoring weight, bed scales have been provided with hospital beds. These beds typically are very expensive. Somewhat less expensive, are portable bed scales, which must be placed under the wheels of mobile hospital beds. These beds and portable scales can only take a single patient weight reading when operated by a staff member.

For monitoring activity and position, video camera systems have been implemented. These systems are often unacceptably intrusive to most patients and provide very limited, non-quantitative information. Their patient activity monitoring capabilities are limited to gross movement. In addition, camera detection of body position is line-of-sight limited. While cameras have the ability to record for extended periods of time, camera systems usually cost in the range of several thousand to tens of thousands of dollars depending upon their degree of deployment, ability to operate in darkness and recording capabilities.

The known solutions fail to provide an integrated monitoring system that can simultaneously measure patient skin pressure, weight, activity, and position. Even those systems that can measure an instance of one or two of these cannot record, playback and trigger alerts continuously. Various combinations of existing systems are extremely expensive and yield inadequate results. Thus a solution is needed that provides continuous monitoring of multiple patient parameters in a single system, particularly for the care of bedridden patients, and that can be used to track and prevent the development, or worsening, of various conditions.

### SUMMARY OF THE INVENTION

The present invention addresses the aforementioned limitations of the prior art by providing a monitoring system for subjects including humans. Such monitoring systems utilize a device that can continuously record data and integrate such information to alarm physicians or care-takers. The monitoring system of the present invention has a variety of uses including, but not limited to, monitoring bedridden patients.

In one aspect, the present invention provides a patient monitoring system for automatically monitoring patient parameters over time while the patient occupies a bed. The patient monitoring system includes a sensing system positioned underneath a patient and separated from the patient by at least one layer of material. The sensing system includes a plurality of sensor cells. The sensor cells automatically collect sensor data related to the patient parameters. An interface is provided for receiving the collected sensor data from the sensing system. A monitoring engine located remotely from the patient receives the collected sensor data from the interface and comprises calculation components for determining the patient parameters from the collected sensor data.

In another aspect, the invention includes a patient monitoring system for automatically and remotely monitoring patient parameters over time for multiple patients, while each of the patients occupies a bed. The patient monitoring system includes multiple sensing systems, each sensing system positioned underneath a patient and separated from the patient by at least one layer of material. Each sensing system includes a plurality of sensor cells, the sensor cells automatically collecting sensor data related to the patient parameters. The monitoring system also includes multiple interfaces, each of the multiple interfaces receiving the collected sensor data from the sensing system and a monitoring engine located remotely from the patients. The monitoring engine receives the collected sensor data from the multiple interfaces and includes calculation components for determining the patient parameters for the multiple patients from the collected sensor data.

In a further aspect of the invention, a patient monitoring method is provided for automatically and remotely monitoring patient parameters over time for multiple patients while each of the patients occupies a bed. The patient monitoring method includes providing multiple sensing systems positioned such that each sensing system is disposed underneath a patient and is separated from the patient by at least one layer of material. Each sensing system includes a plurality of sensor cells. The method includes automatically collecting sensor data related to the patient parameters from said plurality of sensor cells of each sensing system at a remote monitoring engine. The monitoring engine is operatively connected to the multiple sensing systems. The method additionally includes calculating patient parameters using the collected sensor data with calculation components of the monitoring engine.

In yet an additional aspect of the invention, a patient monitoring method is provided for automatically monitoring patient parameters over time for a patient while the patient occupies a bed. The patient monitoring method includes providing a sensing system positioned underneath the patient and separated from the patient by at least one layer of material. The sensing system includes a plurality of sensor cells, the sensor cells automatically collecting sensor data related to the patient parameters. The method additionally includes providing a monitoring engine operatively connected to the sensing system for receiving and processing the collected sensor data. The method further includes calculating patient parameters using the collected sensor data with calculation components of the monitoring engine.

In another aspect, the present invention provides a patient monitoring system for automatically monitoring patient parameters over time while the patient occupies a bed, which includes a sensing system and a monitoring engine. The sensing system is positioned underneath a patient and separated from the patient by at least one layer of material and includes a plurality of sensor cells. The sensor cells automatically collect sensor data related to the patient parameters. The monitoring engine is operatively connected to the sensing system and includes calculation components for determining the patient parameters from the collected sensor data. The collected sensor data include discrete pressure values measured by the sensor cells and which is associated with the locations of the plurality of sensor cells.

The patient monitoring system can also include a display monitor and an interface operatively connected to the sensor cells and the monitoring engine. The interface is configured to integrate the discrete pressure values collected from the sensor cells and to provide the integrated pressure values to the monitoring engine. The calculation components include a pressure distribution determination component.

The monitoring engine is also preferably adapted to generate and display a visual representation of the integrated pressure values on the display monitor in real time.

The monitoring engine preferably includes alarm components for transmitting an alarm signal to indicate the development of a bedsore at one of the locations.

Although illustrative embodiments of the present invention have been described herein with reference to the accompanying drawings, it is to be understood that the invention is not limited to those precise embodiments, and that various other changes and modifications may be effected therein by one skilled in the art without departing from the scope or spirit of the invention.

### BRIEF DESCRIPTION OF THE DRAWINGS

The present invention is described in detail below with reference to the attached drawings figures.

FIG. 1 is a block diagram illustrating an operating environment for a patient monitoring system in accordance with an embodiment of the invention.

FIG. 2 is a block diagram illustrating an operating environment for a patient monitoring system in accordance with another embodiment of the invention.

FIG. 3A is a top plan view illustrating a sensing system in accordance with an embodiment of the invention.

FIG. 3B is a sectional view illustrating a sensing system in accordance with another embodiment of the invention.

FIG. 4 is a block diagram illustrating a monitoring engine in accordance with an embodiment of the invention.

FIG. 5 is a flow chart illustrating a method for patient monitoring in accordance with an embodiment of the invention.

### DETAILED DESCRIPTION OF THE INVENTION

The present invention provides a monitoring system designed to monitor and record patient parameters in real time. In particular, the system can measure pressure values, in addition to conventionally monitored parameters, over an extended period of time, for example, for days or weeks. The pressure values are gathered from discrete known positions for providing a continuous mapping of a pressure distribution of a bed-ridden patient. Rewind and playback functions allow users to rapidly review patient information to diagnose trends and ensure their patients receive the most informed care without the enormous investments of staff time and effort that would be required to compile the information any other way. The patient parameters can include pressure, weight, activity, and position as well as other parameters normally monitored, such as blood pressure and heart rate.

The patient monitoring system combines advances in digital and sensor technology with principles of ergonomics and ease of use. The system combines the basic physical functions of patient skin pressure monitoring, including duration and location of unacceptable pressures, of weighing (which while critical to patient care is neither simple nor easy to achieve in a hospital or clinical setting) with monitoring of activity level and position.

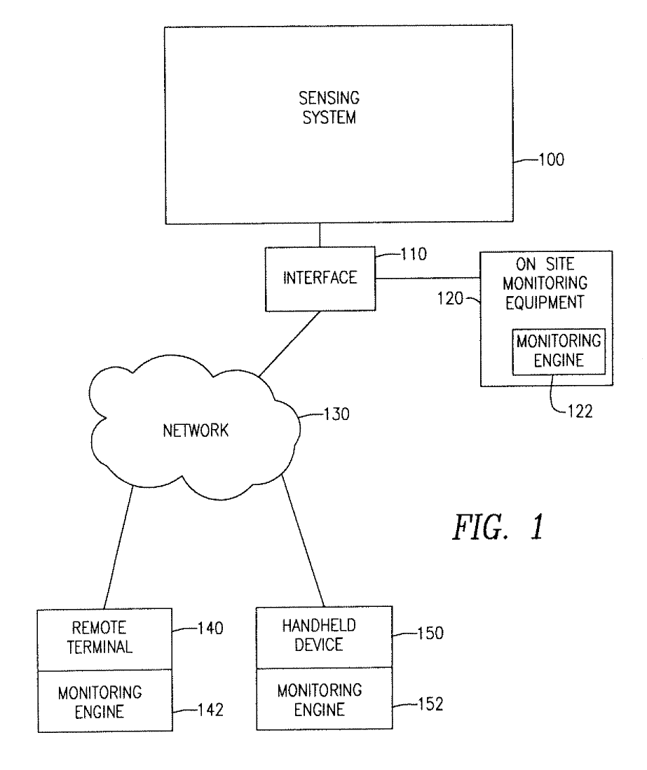

FIG. 1 is a block diagram illustrating an operating environment for a patient monitoring system in accordance with an embodiment of the invention. A sensing system 100 is operably connected with an interface 110. Data is transmitted through the interface 110 to onsite monitoring equipment 120. Data is optionally, or additionally, transmitted from the interface 110 over any suitable network 130 to a remote device. The remote device can include a terminal 140 and/or a handheld device 150. In the embodiment of FIG. 1, the onsite monitoring equipment 120, the remote device (terminal 140 and the handheld device 150) preferably include or are operably connected with monitoring engines 122, 142, 152. Further, it should be understood, that the data collected through the sensing system 100 can be transmitted to fewer devices over the network 130 or to a larger number of devices over the network 130. For example, each staff member attending a patient may have a handheld device 150 that receives data from one or more sensing systems 100.

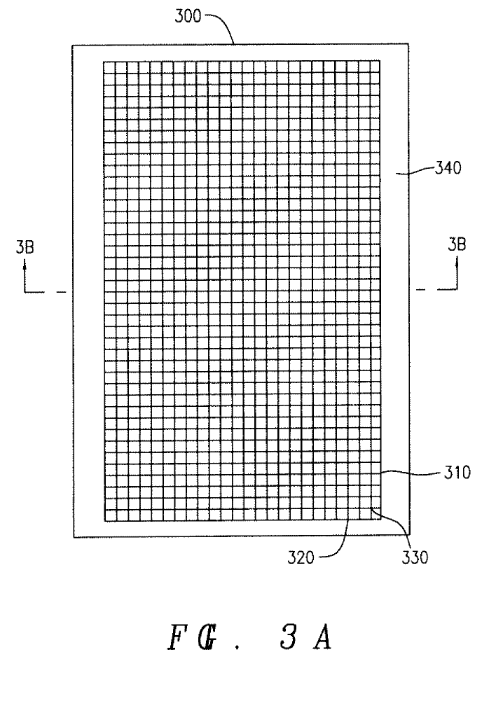

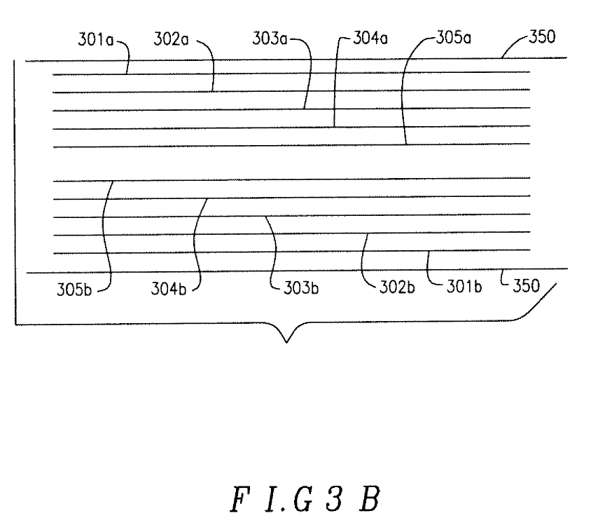

In operation, the sensing system 100 is placed beneath a patient preferably under the bed linens and does not come in contact with the patient. As will be further described below, the sensing system 100 preferably includes a disposable, waterproof cover for protection. Embodiments of the sensing system will be further described below in connection with FIGS. 3A and 3B.

Other monitoring system components shown in FIG. 1, such as the remote terminal 130, the handheld device 150, and the on site monitoring equipment 120 may include computer hardware and software, to be further described below. The computer hardware and software facilitate display of real-time data and recording. The display can occur on a central nursing station, on a hand-held computer or through another patient monitoring system. Data can be transmitted over the Internet from a patient's home to caregivers throughout the world as well. The system is capable of measuring these parameters continuously so multiple patients can be monitored while in their beds, securely and confidentially, from a central nursing station or other location such as a patient's home, or a remote caregiver location. The system also interfaces to industry standard patient monitoring systems, giving these systems a whole new range of insights into the patients being monitored.

The sensor interface 110 may include a snap-on module that rapidly attaches to the sensing system. The interface 110 may include electronics that scan, detect, digitize and wirelessly broadcast the readings gathered from each sensor cell location. In embodiments of the invention, the sensor cells are scanned at a rate of fifteen times per second or more. In one embodiment, the sensor interface 110 connects to a standard USB port. In other embodiments of the invention, the sensor interface is configured to broadcast data wirelessly through any available wireless network. The sensor interface 110 can be powered through the USB port, through a power adapter or long-lasting rechargeable batteries. In embodiments of the invention, the interface processor uses pressure values acquired from the sensor to compute body pressure at various parts of the human subject in real-time.

Other embodiments of the invention include more than one sensor interface 110, or a multi-functional sensor interface 110. For instance, in one embodiment, a sensor interface is implemented for communication with a handheld system that will allow caregivers to go from bed to bed and take readings with maximum efficiency. In another embodiment, a sensor interface plugs into existing bedside patient monitoring systems in order to enhance the power and functions of these systems at a minimum cost. In other embodiments, a sensor interface sends readings wirelessly to central nursing stations so that multiple patients can be monitored simultaneously without requiring visits to the bedside. The electronics in these sensor interfaces may be contained in a small module directly connected to the sensor. The module can be removed from the sensor and reused when replacement of the sensor becomes necessary.

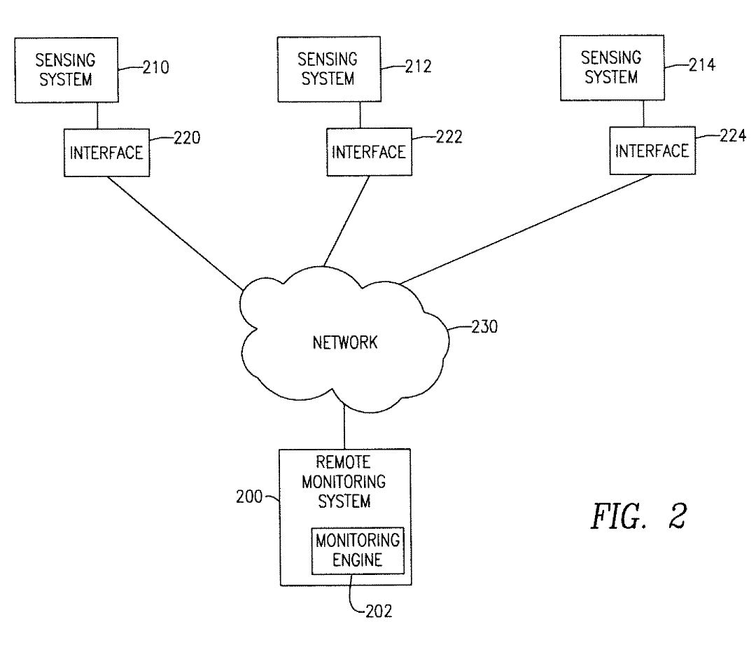

FIG. 2 is a block diagram illustrating an operating environment for a patient monitoring system in accordance with another embodiment of the invention. In the embodiment illustrated in FIG. 2, a remote monitoring system 200 may include a monitoring engine 202 and may be connected over a network 230 with multiple sensing systems 210, 212, 214, through interfaces 220, 222, and 224 respectively. The components shown in FIG. 2 include analogous features to those shown in FIGS. 1 and 2 above.

The components shown in FIGS. 1, 2, and 4 may be or may include a computer or multiple computers. The components may be described in the general context of computer-executable instructions, such as program modules, being executed by a computer. Generally, program modules include routines, programs, objects, components, data structures, etc., that perform particular tasks or implement particular abstract data types.

Those skilled in the art will appreciate that the invention may be practiced with various computer system configurations, including hand-held wireless devices such as mobile phones or PDAs, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote computer storage media including memory storage devices.

The computer system may include a general purpose computing device in the form of a computer including a processing unit, a system memory, and a system bus that couples various system components including the system memory to the processing unit.

Computers typically include a variety of computer readable media that can form part of the system memory and be read by the processing unit. By way of example, and not limitation, computer readable media may comprise computer storage media and communication media. The system memory may include computer storage media in the form of volatile and/or nonvolatile memory such as read only memory (ROM) and random access memory (RAM). A basic input/output system (BIOS), containing the basic routines that help to transfer information between elements, such as during start-up, is typically stored in ROM. RAM typically contains data and/or program modules that are immediately accessible to and/or presently being operated on by processing unit. The data or program modules may include an operating system, application programs, other program modules, and program data. The operating system may be or include a variety of operating systems such as Microsoft Windows® operating system, the Unix operating system, the Linux operating system, the Xenix operating system, the IBM AIX™ operating system, the Hewlett Packard UX™ operating system, the Novell Netware™ operating system, the Sun Microsystems Solaris™ operating system, the OS/2™ operating system, the BeOS™ operating system, the Macintosh™) operating system, the Apache™ operating system, an OpenStep™ operating system or another operating system of platform.

At a minimum, the memory includes at least one set of instructions that is either permanently or temporarily stored. The processor executes the instructions that are stored in order to process data. The set of instructions may include various instructions that perform a particular task or tasks, such as those shown in the appended flowcharts. Such a set of instructions for performing a particular task may be characterized as a program, software program, software, engine, module, component, mechanism, or tool. The patient monitoring system may include a plurality of software processing modules stored in a memory as described above and executed on a processor in the manner described herein. The program modules may be in the form of any suitable programming language, which is converted to machine language or object code to allow the processor or processors to read the instructions. That is, written lines of programming code or source code, in a particular programming language, may be converted to machine language using a compiler, assembler, or interpreter. The machine language may be binary coded machine instructions specific to a particular computer.

Any suitable programming language may be used in accordance with the various embodiments of the invention. Illustratively, the programming language used may include assembly language, Ada, APL, Basic, C, C++, COBOL, dBase, Forth, FORTRAN, Java, Modula-2, Pascal, Prolog, REXX, and/or JavaScript for example. Further, it is not necessary that a single type of instruction or programming language be utilized in conjunction with the operation of the system and method of the invention. Rather, any number of different programming languages may be utilized as is necessary or desirable.

Also, the instructions and/or data used in the practice of the invention may utilize any compression or encryption technique or algorithm, as may be desired. An encryption module might be used to encrypt data. Further, files or other data may be decrypted using a suitable decryption module.

The computing environment may also include other removable/nonremovable, volatile/nonvolatile computer storage media. For example, a hard disk drive may read or write to nonremovable, nonvolatile magnetic media. A magnetic disk drive may read from or writes to a removable, nonvolatile magnetic disk, and an optical disk drive may read from or write to a removable, nonvolatile optical disk such as a CD ROM or other optical media. Other removable/nonremovable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The storage media are typically connected to the system bus through a removable or non-removable memory interface.

The processing unit that executes commands and instructions may be a general purpose computer, but may utilize any of a wide variety of other technologies including a special purpose computer, a microcomputer, mini-computer, mainframe computer, programmed micro-processor, micro-controller, peripheral integrated circuit element, a CSIC (Customer Specific Integrated Circuit), ASIC (Application Specific Integrated Circuit), a logic circuit, a digital signal processor, a programmable logic device such as an FPGA (Field Programmable Gate Array), PLD (Programmable Logic Device), PLA (Programmable Logic Array), RFID processor, smart chip, or any other device or arrangement of devices that is capable of implementing the steps of the processes of the invention.

It should be appreciated that the processors and/or memories of the computer system need not be physically in the same location. Each of the processors and each of the memories used by the computer system may be in geographically distinct locations and be connected so as to communicate with each other in any suitable manner. Additionally, it is appreciated that each of the processor and/or memory may be composed of different physical pieces of equipment.

A user may enter commands and information into the computer through a user interface that includes input devices such as a keyboard and pointing device, commonly referred to as a mouse, trackball or touch pad. Other input devices may include a microphone, joystick, game pad, satellite dish, scanner, voice recognition device, keyboard, touch screen, toggle switch, pushbutton, or the like. These and other input devices are often connected to the processing unit through a user input interface that is coupled to the system bus, but may be connected by other interface and bus structures, such as a parallel port, game port or a universal serial bus (USB).

One or more monitors or display devices may also be connected to the system bus via an interface. In addition to display devices, computers may also include other peripheral output devices, which may be connected through an output peripheral interface. The computers implementing the invention may operate in a networked environment using logical connections to one or more remote computers, the remote computers typically including many or all of the elements described above.

Various networks may be implemented in accordance with embodiments of the invention, including a wired or wireless local area network (LAN) and a wide area network (WAN), wireless personal area network (PAN) and other types of networks. When used in a LAN networking environment, computers may be connected to the LAN through a network interface or adapter. When used in a WAN networking environment, computers typically include a modem or other communication mechanism. Modems may be internal or external, and may be connected to the system bus via the user-input interface, or other appropriate mechanism. Computers may be connected over the Internet, an Intranet, Extranet, Ethernet, or any other system that provides communications. Some suitable communications protocols may include TCP/IP, UDP, or OSI for example. For wireless communications, communications protocols may include Bluetooth, Zigbee, IrDa or other suitable protocol. Furthermore, components of the system may communicate through a combination of wired or wireless paths.

Although many other internal components of the computer are not shown, those of ordinary skill in the art will appreciate that such components and the interconnections are well known. Accordingly, additional details concerning the internal construction of the computer need not be disclosed in connection with the present invention.

FIG. 3A is a top plan view illustrating a sensing system 300 in accordance with an embodiment of the invention. The sensing system 300 includes multiple electrodes. In one embodiment, the system 300 includes sets of preferably perpendicularly disposed intersecting electrodes. As illustrated in the embodiment of FIG. 3A, a set of horizontally disposed electrodes 310 intersects with a set of vertically disposed electrodes 320, thus creating multiple intersections 330. This sensing system configuration is merely exemplary, and any suitable sensing configuration, such as those disclosed in U.S. Pat. Nos. 5,033,291, 4,856,993, and 4,734,034 may be implemented.

FIG. 3B is a sectional view illustrating a sensing system in accordance with an embodiment of the invention. Outermost layers 301a and 301b designate a backing sheet material. The backing sheet material 301a and 301b is removable and is preferably positioned adjacent a disposable covering 340 for the sensing system. Conducting layers 302a and 302b are preferably applied to each of the backing sheets. Dielectric layers 303a and 303b are disposed adjacent the conducting layers 303a and 303b. Conductive stripes 304a and 304b are disposed adjacent the dielectric layers 303a and 303b. Stripes of pressure sensitive material 305a and 305b are applied adjacent conductive stripes 304a and 304b.

In one embodiment, the sensing system 300 includes a thin, flexible sensor, which extends the length and width of a standard hospital mattress (typically the size of a twin bed). A suitable sensor thickness is employed, as easily determined by one skilled in the art. In one preferred embodiment, the sensor's thickness is approximately ⅛ inch. This thin and flexible sensing system can cover the surface of a hospital bed and can be placed under the bed-sheets so as not to come into contact with the patient. In alternative embodiments, if desired, the sensor comes into contact with the patient.

As illustrated above, the sensor contains sensing elements arranged in rows and columns. Their size and spacing are preferably optimized for the measuring of weight, position, pressure and activity. In embodiments of the invention, approximately three hundred sensor cells are distributed over the surface area of the sensing system. These sensors, as large as a twin bed, ⅛ of an inch thick or less and containing about one sensor cell per square inch, combined with the monitoring components described below, are able to continuously monitor patients in their beds for long periods of time.

Preferably, each sensor cell scans at a rate of about fifteen times per second or more. Alternatively, each sensor cell scans at a rate of about twenty five times per second or more. Other scanning rates are also possible. Preferably, the sensor can be used for multiple patient stays and can be in continuous use for up to two years or more. While long-lasting, the sensors are affordable and easily replaced.

In embodiments of the invention implementing a cover, the cover is a waterproof cover that is preferably a disposable plastic sensor cover. Other materials may alternatively be implemented to construct the sensor cover. The cover may be implemented to keep the sensor clean, to protect the sensor, and ensure that cross-contamination does not occur. Sensor covers are easily placed in the hospital room without tools or the need for training. The disposable sensor covers also eliminate the need for sensor replacement solely due to contamination. In embodiments of the invention, the disposable cover is replaced with each new patient or even during a patient's stay if required.

In one embodiment, the sensor is used for multiple patient stays. It may be in continuous long term use, for example for up to two years. Thus, in a preferred embodiment, the sensing system comprises a bed-size sheet that does not come into direct contact with the patient. The system is non-intrusive, requires no adjustment by medical professionals, and can operate for the entirety of a patient's stay without intervention.

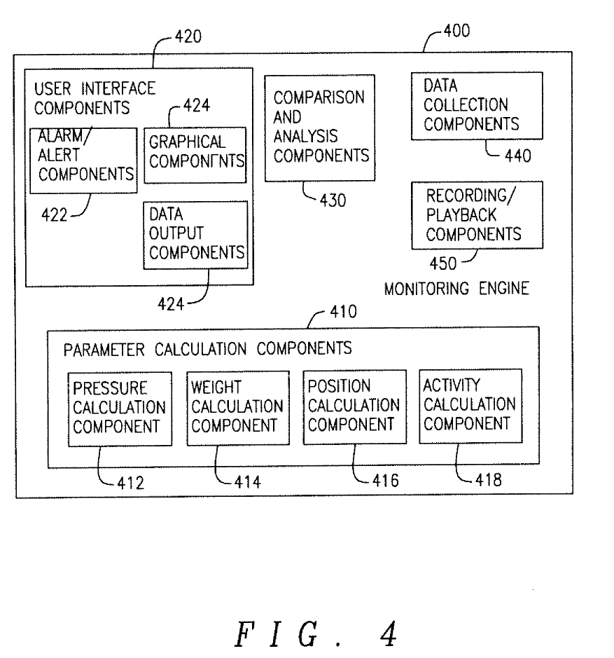

FIG. 4 is a block diagram illustrating a monitoring engine 400 in accordance with an embodiment of the invention. The monitoring engine 400 is implemented by a computer processor and may be stored in a computer memory. The monitoring engine 400 is implemented on any and all of the remote terminal, handheld devices and on-site monitoring equipment shown in FIG. 1 and on the remote terminal shown in FIG. 2.

The monitoring engine 400 includes data collection components 440 for receiving data from the sensing system and interface described above. The collected data is processed by parameter calculation components 410. The parameter calculation components 410 include a pressure calculation component 412, a weight calculation component 414, a position calculation component 416, and an activity calculation component 418. These calculation or determination components utilize the collected sensor data and transform the data to determine the desired parameters in a manner known to those skilled in the art. For instance, to determine activity, the activity calculation component 418 determines changes in pressure on various sensor cells over time. The position calculation component 416 relates pressure values over various sensor cells. The weight calculation component 414 determines overall pressure measurements as well as increases and decreases in overall pressure over time. In other embodiments of the invention, in addition to patient parameters such as pressure, weight, activity, and position, other patient parameters, such as respiration, temperature, and heart rate may also be monitored.

Pressure measurements provided by the sensors are particularly useful for prevention of bedsores. A bedsore relates to breakdown in skin due to prolonged application of pressure. Based on sensor data, the monitoring engine 400 provides a location of forming bedsores for a bed-bound patient in real time. As will be further described below, pressure values beyond a predetermined threshold may trigger an alarm or other indicator that provides an indication of the forming bedsore. Furthermore, based on the data provided to the monitoring engine from the sensing system, the patient monitoring system provides a location of the forming bedsore. The impact of the system for preventive care is therefore considerable

In embodiments of the invention, the monitoring engine further includes comparison and analysis components 430. The comparison and analysis components 430 compare measured or calculated values to stored threshold values or profiles. Furthermore, the comparison and analysis components 430 enable creation and comparison of individual patient files. The comparison and analysis components 430 help to create trend graphs. Optimally, the trend graphs reveal information such as body weight of various body parts over time. Suitable data output formats may be used, including but not limited to, the output of data in Excel or other spreadsheet format to allow for off-line analysis.

Recording and playback components 450 enable recordings to be made and played back to hospital or care-giving personnel. Rewind and playback functions allow users to rapidly review patient information to diagnose trends and ensure their patients receive the most informed care without the enormous investments of staff time and effort that would be required to compile the information any other way.

User interface components 420 facilitate interpretation of monitored data and preferably include alarm/alert components 422, graphical components 424, and data output components 426.

In embodiments of the invention, the user interface components 420 includes PC-based software that allows users to display sensor readings in color and 3D graphical displays in real-time. The user interface components 420 create easy-to-use trend graphs (of weight for example) and facilitate the output of data in Excel or other spreadsheet format to facilitate off-line analysis.

The alarm/alert components 422 are triggered if unacceptable conditions occur. Such conditions may include a patient leaving the patient bed, pressures of problematic degree and duration, hyperactivity, etc. For instance, when connected to a WAN, one nursing station monitors dozens of sensing systems and display the patients' conditions with intuitive green/yellow/red indicators. For example, these indicators may be used for problem weight trends, excessive pressure, excessive movement, and out-of-bed alarms. In embodiments of the invention, audible and voice-synthesized alarms are also provided.

The monitoring engine 400 enables interfacing to standard patient monitoring systems, eliminating the need for a dedicated computer. The monitoring system allows direct connection of the sensor interface to various standard patient monitoring systems, thus reducing the amount of hardware necessary to use the monitoring system for healthcare centers that already own a patient vital signs monitoring system.

When a dedicated computer is implemented, such as for a handheld device or remote terminal, embodiments of the invention implement a standard Wintel PC, desktop, laptop, Pocket PC, or other type of computing device as described above. Preferably the components are linked to a color display.

In a preferred embodiment of the invention, the monitoring engine 400 supports HIPAA-compliant network and Internet connections, allowing remote network and Internet-based monitoring of patient real-time data, alerts and alarms and pre-recorded results. Using this feature, a single computer can monitor multiple, remote sensing systems with a minimum of hardware or staff attention.

The monitoring system has been designed to monitor and record patient information over extended periods of time, such as for days or weeks if necessary. In this way, physicians and caregivers receive an unparalleled perspective on their patients' health.

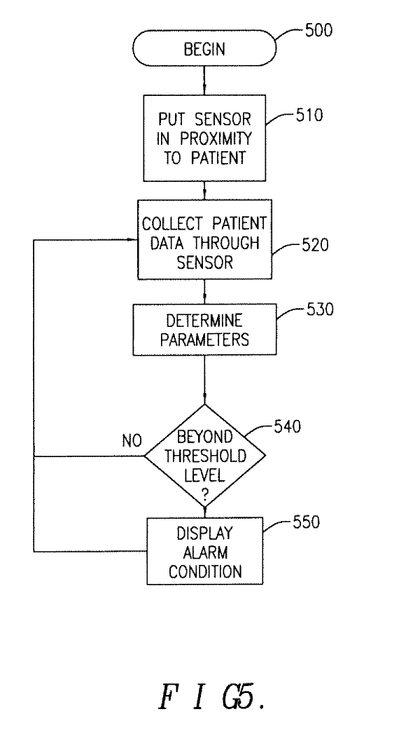

FIG. 5 is a flow chart illustrating a method for patient monitoring in accordance with an embodiment of the invention. The method begins at step 500 and a sensing system is put in proximity to the patient 510. The monitoring engine collects patient data through a sensing system in step 520. Typically this data will be pressure data recorded at different locations and times. The monitoring system then determines parameters 530. The parameters may include for example, pressure, weight, activity, and position. Other parameters may also be monitored. The system may contain stored threshold levels or stored profiles for comparison for these parameters and in step 540, the monitoring engine may determine if the determined parameters are beyond a predetermined threshold level. If the determined parameters are not beyond the threshold level, the monitoring engine continues with monitoring in step 520. If the parameters are beyond the threshold level, the monitoring engine displays or sounds an alarm condition in step 550, and again continues monitoring. In the case of position parameters, instead of a threshold level, the system may store a number of predetermined acceptable and unacceptable positions and compare the determined positions to the stored positions. In the case of weight parameters, the system may detect weight gain or loss and may utilize the most recent weight determination to identify an appropriate dose of medication.

The simplicity of the monitoring system makes it convenient for patients to operate and connect to their Internet-connect PC's without professional assistance. This allows doctors and nurses to monitor risk of pressure sores, patient weight, activity levels, restlessness when awake or asleep, frequency of movement and presence in bed anytime and anywhere.

In accordance with the aspects of the present invention, the monitoring system is suitable for use by patients with varying conditions. Treatment of certain prevalent patient conditions particularly benefits from the features of the monitoring system. These conditions are summarized in the table below. Pressure sores, for example, represent 1 million cases per year alone. Conditions such as cancer can expose the patient to a high pressure ulcer risk.

| | | | | | | | Long- | | |
|---|---|---|---|---|---|---|---|---|---|
| | | Bed | | | | | Term | HI | |
| | | Confinement | Weight | Activity | Pressure | Position | Recording | Treatment | Status |
| No. | Conditions | Required | Critical | Critical | Critical | Critical | Critical | cost | Available |
| 1 | Decubitus | Yes | | Yes | Yes | Yes | Yes | Yes | Yes |
| | Ulcers | | | | | | | | |
| | (Pressure or | | | | | | | | |
| | Bedsores) | | | | | | | | |
| 2 | Paralysis | Yes | Yes | Yes | Yes | Yes | Yes | Yes | Yes |
| 3 | Post-Op | Yes | | Yes | Yes | Yes | Yes | Yes | Yes |
| | Orthopedic | | | | | | | | |
| | Surgery | | | | | | | | |
| | Patients | | | | | | | | |
| 4 | Paralytic | Yes | Yes | Yes | Yes | Yes | Yes | Yes | Yes |
| | Stroke (length | | | | | | | | |
| | of stay 28 | | | | | | | | |
| | days) | | | | | | | | |
| 5 | Non- | Yes | Yes | Yes | Yes | Yes | Yes | Yes | Yes |
| | Ambulatory | | | | | | | | |
| | Alzheimer's | | | | | | | | |
| 6 | Organ | Yes | Yes | Yes | Yes | Yes | Yes | Yes | Yes |
| | Transplant | | | | | | | | |
| | Patients | | | | | | | | |
| 7 | Pre-Eclampsia | Yes | Yes | Yes | Yes | | Yes | Yes | Yes |
| | (pregnancy | | | | | | | | |
| | hypertension) | | | | | | | | |
| 8 | Sleep Disorder | Yes | | Yes | | Yes | Yes | | Yes |
| | Diagnosis | | | | | | | | |
| 9 | Thoracic | Yes | Yes | Yes | Yes | | Yes | Yes | Yes |
| | Surgery | | | | | | | | |
| 10 | Total Knee | Yes | | Yes | Yes | Yes | Yes | Yes | Yes |
| | Replacements | | | | | | | | |
| 11 | CABG | Yes | Yes | Yes | Yes | Yes | Yes | Yes | Yes |
| | (Coronary | | | | | | | | |
| | Artery Bypass | | | | | | | | |
| | Graft | | | | | | | | |
| | Surgery) | | | | | | | | |
| 12 | Malignanto | Yes | Yes | Yes | Yes | Yes | Yes | Yes | Yes |
| | Neoplasms | | | | | | | | |
| | (avg length of | | | | | | | | |
| | stay: 7-8 | | | | | | | | |
| | days) | | | | | | | | |

Conditions that are relevant to the present monitoring system include: i) skin pressure is important or risk of bedsores is high; and ii) requires bed-rest or confinement to a bed for significant periods of time. These two conditions do not permit patients to get out of bed to be weighed and expose the patient to complications of restricted activity and bed confinement. Other conditions relevant to the present monitoring system include situations in which: iii) monitoring patient weight is critical; iv) monitoring patient in-bed activity level is critical; v) monitoring patients' positions in bed is important; vi) “long-term” (hours to days) recording of bed activity or pressures is required; and vii) high treatment or consequence cost (such as bedsores).

One outcome of healthcare's inability to continuously monitor the physical condition of patients, for example, is the incidence of decubitus ulcers (bed or pressure sores). The problem impacts approximately one million patients a year and costs healthcare billions of dollars per year and the disclosed monitoring system can present a solution.

In addition to the capability to provide real-time monitoring and recording of patient skin pressure, weight, position, and activity level while in bed, the system offers valuable economic benefits by increasing the number of patients that can receive outstanding and improved care from existing staff. Many hospitals and extended care facilities are confronted with the problem of having too few staff members to monitor patients adequately. As a result, many patients each year fall from bed, are injured in bed-related accidents, are misdiagnosed, improperly medicated, suffer pressure sores or otherwise suffer from inadequate monitoring.

In accordance with aspects of the present invention, the present monitoring system connects to an existing patient monitoring system. The present software and system modules are available to allow direct connection of the sensor interface to various standard patient monitoring systems. This conveniently reduces the amount of hardware necessary to use the present monitoring system for healthcare centers that already own a patient vital signs monitoring system. In addition, it integrates the sensor-related patient metrics to other monitoring data.

The monitoring system disclosed herein is capable of continuously monitoring patients and alerting caregivers when threatening conditions exist. The system also makes long-term recordings for later review. Monitoring and recording these parameters can be critical factors in effective patient care.

While particular embodiments of the invention have been illustrated and described in detail herein, it should be understood that various changes and modifications might be made to the invention without departing from the scope and intent of the invention. It is also understood that certain features and sub-combinations are of utility and may be employed without reference to other features and sub-combinations. This is contemplated and within the scope of the appended claims.

What is claimed is:

1. A patient monitoring system for automatically monitoring patient parameters over time while the patient occupies a bed, the patient monitoring system comprising:
a sensing system positioned underneath a patient and separated from the patient by at least one layer of material, the sensing system comprising a plurality of sensor cells, the sensor cells automatically collecting sensor data related to the patient parameters;
an interface for receiving the collected sensor data from the sensing system; and
a monitoring engine located remotely from the patient, the monitoring engine receiving the collected sensor data from the interface and comprising calculation components for determining the patient parameters from the collected sensor data.

2. The patient monitoring system of claim 1, said patient monitoring system further comprising a display monitor, and wherein the monitoring system includes a pressure distribution determination component, wherein said collected sensor data include discrete pressure values associated with the location of said sensor cells, wherein the interface is configured to integrate the discrete pressure values, and said monitoring engine is configured to generate and display a visual representation of the integrated pressure values on said display monitor in real time.

3. The patient monitoring system of claim 2, wherein the monitoring engine comprises alarm components for transmitting an alarm signal to indicate when the patient begins to develop a bedsore.

4. The monitoring system of claim 1, wherein the monitoring engine comprises an activity determination component for determining a level of patient activity from the collected sensor data.

5. The monitoring system of claim 1, wherein the monitoring engine comprises a position determination component for determining a patient position based on the collected sensor data.

6. The monitoring system of claim 1, wherein the monitoring engine comprises a weight determination component for monitoring a patient weight based on collected sensor data.

7. The monitoring system of claim 1, further comprising a cover for protecting the sensing system.

8. The monitoring system of claim 7, wherein the cover for protecting the sensing system is a removable waterproof cover.

9. The monitoring system of claim 1, wherein said sensing system contains sensing elements arranged in rows and columns and the sensing system contains between 250 and 350 sensor cells.

10. The monitoring system of claim 1, wherein said sensing system has a thickness of about ⅛ inch.

11. The monitoring system of claim 1, wherein said sensor cell scans at a rate of about 15 times per second or more.

12. The pressure distribution measuring system of claim 1, wherein said sensor cell scans at a rate of about 25 times per second or more.

13. The monitoring system of claim 1, wherein the interface is connected to a standard USB port.

14. The monitoring system of claim 1, wherein the interface includes wireless transmission components.

15. The monitoring system of claim 1, wherein the monitoring engine functions in conjunction with at least one of a bedside patient monitoring system, a handheld PDA, and a nursing station computer.

16. A patient monitoring system for automatically and remotely monitoring patient parameters over time for multiple patients, while each of the patients occupies a bed, the patient monitoring system comprising:
multiple sensing systems, each sensing system positioned underneath a patient and separated from the patient by at least one layer of material, each sensing system comprising a plurality of sensor cells, the sensor cells automatically collecting sensor data related to the patient parameters;
multiple interfaces, each of the multiple interfaces for receiving the collected sensor data from the sensing system; and
a monitoring engine located remotely from the patients, the monitoring engine receiving the collected sensor data from the multiple interfaces and comprising calculation components for determining the patient parameters for the multiple patients from the collected sensor data.

17. The patient monitoring system of claim 16, further comprising a display monitor, wherein the monitoring system provides a pressure distribution determination component, wherein each sensor cell measures a discrete pressure value, the interface collects and integrates the pressure values from the sensor cells, and the monitoring engine displays a visual representation of the integrated pressure values on the display monitor in real time.

18. The patient monitoring system of claim 17, wherein the monitoring engine comprises alarm components for transmitting an alarm signal to indicate when the patient begins to develop a bedsore.

19. The monitoring system of claim 16, wherein the monitoring engine comprises an activity determination component for determining a level of patient activity from the collected sensor data.

20. The monitoring system of claim 16, wherein the monitoring engine comprises a position determination component for determining a patient position based on the collected sensor data.

21. The monitoring system of claim 16, wherein the monitoring engine comprises weight determination component for monitoring a patient weight based on collected sensor data.

22. The monitoring system of claim 16, further comprising a cover for protecting the sensing system.

23. The monitoring system of claim 22, wherein the cover for protecting the sensing system is a removable waterproof cover.

24. The monitoring system of claim 16, further comprising multiple monitoring engines, wherein at least one monitoring engine is located at a patient site and at least one monitoring engine is located on a handheld computing device.

25. The monitoring system of claim 16, wherein the monitoring engine is located remotely from the patient and the interface wirelessly transmits the collected sensor data to the monitoring engine.

26. A patient monitoring method for automatically and remotely monitoring patient parameters over time for multiple patients, while each of the patients occupies a bed, the patient monitoring method comprising:
providing multiple sensing systems positioned such that each sensing systems is disposed underneath a patient and is separated from the patient by at least one layer of material, each sensing system comprising a plurality of sensor cells;
automatically collecting sensor data related to the patient parameters from said plurality of sensor cells of each sensing system at a remote monitoring engine operatively connected to said multiple sensing systems; and
calculating patient parameters using the collected sensor data with calculation components of the monitoring engine.

27. The monitoring method of claim 26, further comprising implementing the sensor cells for measuring a pressure value at multiple locations underneath the patient.

28. The monitoring method of claim 27, further comprising comparing the measured pressure values to threshold values to determine if the threshold values have been exceeded.

29. The monitoring method of claim 28, further comprising generating an alarm if the threshold pressure values have been exceeded.

30. The monitoring method of claim 26, further comprising determining activity patterns from the collected sensor data.

31. The monitoring method of claim 30, further comprising determining if the activity pattern exceeds an activity threshold.

32. The monitoring method of claim 31, further comprising generating an alarm if the activity threshold has been exceeded.

33. The monitoring method of claim 26, further comprising determining a patient position from the collected sensor data.

34. The monitoring method of claim 33, further comprising comparing the patient position to dangerous patient positions.

35. The monitoring method of claim 34, further comprising generating an alarm if the patient position is a dangerous patient position.

36. A patient monitoring method for automatically monitoring patient parameters over time for a patient while the patient occupies a bed, the patient monitoring method comprising:
providing a sensing system positioned underneath the patient and separated from the patient by at least one layer of material, the sensing system comprising a plurality of sensor cells, the sensor cells automatically collecting sensor data related to the patient parameters;
providing a monitoring engine operatively connected to said sensing system for receiving and processing the collected sensor data; and
calculating patient parameters with calculation components of the monitoring engine.

37. The monitoring method of claim 36, further comprising implementing the sensor cells for measuring a pressure value at multiple locations underneath the patient.

38. The monitoring method of claim 37, further comprising comparing the measured pressure values to threshold values to determine if the threshold values have been exceeded.

39. The monitoring method of claim 38, further comprising generating an alarm if the threshold pressure values have been exceeded.

40. The monitoring method of claim 36, further comprising determining activity patterns from the collected sensor data.

41. The monitoring method of claim 40, further comprising determining if the activity pattern exceeds an activity threshold.

42. The monitoring method of claim 41, further comprising generating an alarm if the activity threshold has been exceeded.

43. The monitoring method of claim 36, further comprising determining a patient position from the collected sensor data.

44. The monitoring method of claim 43, further comprising comparing the patient position to dangerous patient positions.

45. The monitoring method of claim 44, further comprising generating an alarm if the patient position is a dangerous patient position.

46. A patient monitoring system for automatically monitoring patient parameters over time while the patient occupies a bed, the patient monitoring system comprising:
a sensing system positioned underneath a patient and separated from the patient by at least one layer of material, the sensing system comprising a plurality of sensor cells, the sensor cells automatically collecting sensor data related to the patient parameters; and
a monitoring engine operatively connected to said sensing system, the monitoring engine comprising calculation components for determining the patient parameters from the collected sensor data, wherein said collected sensor data include discrete pressure values measured by said sensor cells and associated with the locations of said plurality of sensor cells.

47. The patient monitoring system of claim 46, said patient monitoring system further including a display monitor and an interface operatively connected to the sensor cells and the monitoring engine, said interface configured to integrate the collected discrete pressure values and provide the integrated pressure values to the monitoring engine, wherein said calculation components include a pressure distribution determination component, and wherein the monitoring engine is adapted to generate and display a visual representation of the integrated pressure values on said display monitor in real time.

48. The patient monitoring system of claim 46, wherein the monitoring engine comprises alarm components for transmitting an alarm signal to indicate the development of a bedsore at one of the locations.
